(19) United States
(12) Patent Application Publication
(10) Pub. No.: US 2018/0256657 A1
(43) Pub. Date: Sep. 13, 2018

(54) Natural method for eliminating HIV to be employed by type 1 and 2 HIV sufferers

(71) Applicant: Denessa Rochelle Luckett, Indianapolis, IN (US)

(72) Inventor: Denessa Rochelle Luckett, Indianapolis, IN (US)

(21) Appl. No.: 15/456,515

(22) Filed: Mar. 11, 2017

(51) Int. Cl. A61K 36/07; A61K 36/48; A61K 36/54; A61K 36/28; A61K 36/42; A61K 36/886; A61K 36/31; A61K 36/736; A61K 36/81; A23L 33/10

(52) U.S. Cl. CPC A61K 36/07; A61K 36/48; A61K 36/54; A61K 36/28; A61K 36/42; A23V 2002/00; A61K 36/31; A61K 36/736; A61K 36/81; A23L 33/10; A61K 36/886

(57) ABSTRACT

A defined natural procedure that lists a series of steps that will effectively eliminate HIV from the victim's body in 2 months. The procedure starts with prayer to the God of Abraham for healing which continues throughout the process. It includes treating the yeast infection with a physician prescribed antifungal product and prednisone 7-day step down regimen (if needed). The elimination of refined sugar products and the inclusion of daily cardiovascular and weight bearing exercise will cut off the HIV's food supply. The final steps involve performing daily internal cleanse to detoxify the body, abstaining from sexual intimacy, and consuming a combination of foods in the form of a salad for breakfast with the remaining meals calorie restricted. With the HIV virus eliminated from the victim's body, the immune system will rebuild/replenish itself to the point where it can effectively combat opportunistic diseases.

### PRIOR ART

| Cited Patent | Filing date | Publication date | Applicant | Title |
|---|---|---|---|---|
| WO2012176215 A1 | Jun. 11, 2012 | Dec. 27, 2012 | Shinhasan Manohar | A plant based antiviral |
| | | | Pundaleekappa | composition for the |
| | | | | treatment of hiv and |
| | | | | hiv related acquired |
| | | | | immune deficiency |
| | | | | syndrome |
| WO2016156920 A1 | Mar. 31, 2015 | Oct. 6, 2006 | Seyyed Milad | Producing herbal |
| | | | Abrishamifar, Melika | medicine with |
| | | | Yousefi | mosquito stomach |
| | | | | enzyme to treat virus |
| | | | | and aids |

### FEDERALLY SPONSORED RESEARCH OR DEVELOPMENT

N/A

### PARTIES TO A JOINT RESEARCH AGREEMENT

N/A

### INCORPORATION-BY-REFERENCE OF MATERIAL

N/A

### STATEMENT REGARDING PRIOR DISCLOSURES BY THE INVENTOR

N/A

### BACKGROUND OF THE INVENTION

### Field of the Invention

Current treatments for HIV involve retroviral drugs that disrupt viral replication long enough to allow the HIV sufferer's immune system time to recover its T-cell count to a sufficient level to help the body fight opportunistic diseases. This treatment and similar treatments manages the virus throughout the remainder of the victim's life rather than eliminating the virus entirely. The strategy is to disrupt viral replication which decreases the virus's destructive attack against the victim's T-cells. Maintaining T-cells at normal levels helps the victim's immune system to effectively fight opportunistic diseases found in AIDS. This form of treatment strategy places a tremendous burden on a nation's healthcare system. Those without access to healthcare die from the ravages of the onslaught of opportunistic diseases which results from a weakened immune system's inability to fight off disease.

### Description of Related Art

WO2012176215 A1 describes “a plant based antiviral composition for the treat of HIV and HIV related acquired immunodeficiency syndrome” that kills the HIV through the use of plant-based holy basil injectable preparation. In order to extract the active ingredient from holy basil (Ocimum Sanctum), a number of chemical reagents are needed as well as the use of the skills of a chemist. The isolated preparation is an injection that would have to be administered by a medical professional. This procedure does not empower the HIV suffer to care for themselves.

WO2016156920 A1 describes “producing herbal medicine with mosquito stomach enzyme to treat virus and aids by mixing: leaf and root of cranesbill, basil, mint, date, the enzyme generated in the stomach of mosquito” to kill HIV. This preparation, like the one described above uses holy basil along with other ingredients. This preparation also requires the skills of a chemist to isolate active ingredients and the skills of a biologist to extract, isolate, and purify the enzymes from the mosquito's stomach. This procedure does not empower the HIV sufferer.

My patent will describe a procedure to eliminate the HIV virus entirely, eliminate AIDs (restore the body's immune system so that it can effectively fight diseases), and eliminate the financial burden that the current mainstream life-time treatments for HIV/AIDs places on the health care system. Over 95% of the treatment described herein can be employed by the HIV suffer.

### BRIEF SUMMARY OF THE INVENTION

A series of procedures as well as the consumption of a specific combination of foods consumed for a 2 month period will sufficiently eliminate the types 1 and 2 HIV virus and restore the body's immune system to fight off opportunistic diseases (AIDs). With the exception of one step, all of the prescribed procedures can be performed alone by the HIV sufferer.

### BRIEF DESCRIPTION OF THE SEVERAL VIEWS OF THE DRAWING(S)

N/A

### DETAILED DESCRIPTION OF THE INVENTION

1. First and foremost the HIV sufferer should pray to the God of Abraham for healing, as well as through the administration of the remaining procedures.
A. If the HIV sufferer hasn't participated in risky behaviors that would lead to infection, one should
   Pray: Should I suffer as an evil doer?
B. If the HIV sufferer has participated in behaviors that exposed them to the disease, Pray: Be merciful to me a sinner and please heal me. Help me to go and sin no more.

2. Have physician treat the yeast infection with the normal fungal infection medication and, if needed, to prescribe 7-day step prednisone (Day 1: 7 prednisone tabs Day 2: 6 prednisone, etc). This step should occur before embarking on the remaining steps.

3. Steps 1, 3-8 should be performed daily for at least 2 months

4. Eliminate refined sugar from diet. This step may take a while depending on the level of addiction, but it is absolutely necessary to perfect this step before advancing to other steps. Eliminate the following:
A. Store bought fruit juices
B. Soda, sweetened beverages
C. Candy, cookies, brownies, donuts, pies, cakes, ice cream, flavored yogurt, bread; high glycemic foods such as potatoes, corn, etc should be kept to a minimum

5. Non-strenuous exercise
A. 20-30 cardio workout, aerobic breathing (meaning you are not out of breathe while working out) 5-6 days per week
B. Walk 20-30 minutes per day
C. Perform mild weight lifting 3-4 days a week. Work upper and lower body on alternating days. Use exercise machines that work the upper body: chest press, triceps bar, biceps bar, and lat pull-down performing 2 sets of 10 repetitions on each machine at a light to moderate level where each set can be completed correctly without strain. On alternating days use exercise machines that work the lower body such as the seated leg press performing 3 sets of 10 and abdominal crunch machine to work the abdominals and obliques performing 2 sets of 15 at a light to moderate level.

6. Abstain from sexual intercourse

7. Perform internal cleanse to detoxify the body:
A. Hydrate, drink water throughout the day (drink half your body weight in oz of water)
B. On a daily basis prior to eating, administer a water enema after eliminating waste naturally. Replace one water enema with organic coffee enema (follow steps 7.B. a-g except add 1-2 TBSP of ground organic coffee to the water, strain heated water through a fine mesh stainless steel strainer).
   a. Heat purified (purchase water from a grocery store water purification system such as Culligan) water to boiling, allow to cool to ˜98-105 F
   b. Measure the water temperature with a candy thermometer
   c. Fill an enema bag (1.6 L capacity) 85% full with the heated water
   d. Lubricate the probe with oil (such as olive oil)
   e. Remove air from the tube by allowing water to flow through the tube, re-clamp
   f. Lie on left side, insert probe, and modulate the fluid entry to tolerable flow until most of the water is inside the body. Leave some water in the enema bag to prevent the introduction of air into the body
   g. Allow the water to remain in body for 20 minutes, lie on each side of the body 5 minutes—use a timer. The time introducing the water on the right side will usually take ˜2 minutes, remain on the left side for the balance of the 5 minutes, lie on your back, then your right side, and lie on your stomach last (if too uncomfortable to lie on your stomach, bend right leg slightly and use as a prop to raise your stomach a few inches off floor). In the beginning, you may not be able to hold the water for the entire 20 minutes. The 20 minutes is a target.
   h. Eliminate the water (allow 5-15 minutes to complete the successive eliminations)

8. Diet
A. Lunch and dinner should be sensible basic meal that include meat protein and low-glycemic vegetables; avoid eating from 8 p.m. to 4 a.m. unless hungry
B. Snacks (some fresh fruit or raw non-starch vegetables)
C. Breakfast (it is critical that this meal is eaten daily as the first meal of the day)
   a. Sprouts: advanced planning is required for this step. Fenugreek seeds (Locate at an Indian grocer) and brown lentils will be sprouted separately.
      i. Add about ⅛ cup of fenugreek seeds to a bowl and ¼ cup of brown lentils to another. Soak the seeds in water over night.
      ii. Pour off the water and cover with a cheese cloth during the day or leave open to the air
      iii. Rinse the seeds every day
      iv. Eventually the seeds will sprout. Depending on the season this could take 3-5 days to reach the point where they are edible.
      v. Make new batches as needed as this is an ingredient that is needed daily. Do not over produce the sprouts because the longer sprouted seeds grow, the more inedible they become
   b. Add a handful of fenugreek and lentil sprouts to your salad bowl
      Seeds—freshly grounded and whole
   c. Freshly grounded—grind in a coffee grinder
      1 TBSP Black sesame seeds and 2 TBSP flax* (unplug the grinder, use a spoon to scoop out the ground seeds, add to ½ mashed avocado and mix thoroughly)
   d. Whole seeds
      Shelled sunflower and in-shell pumpkin seeds (˜1-2 TBSP each or to your taste)
   e. Aloe Vera Barbadensis (locate whole fresh leaf at Mexican grocer) It is preferable to buy leaves that are whole, unbroken, no browned spots or gashes. Wash the leaf. Cut off ˜2 inches. Cut the prickly sides off the piece, then slice it open preserving as much of the gel as possible. Cut the gel off the remaining slice, and cut it into ½ inch chunks. Add to salad. (Note, the gel is slimy, take care not to cut yourself) Wrap the remaining leaf with plastic wrap or aluminum foil and refrigerate for next use.
   f. Watercress leaves—add half of the bunch of pre-cleaned leaves with stems which should amount to at least a cup. But you can add as much as you like.
   g. Add fresh Shiitake (Reishi or Maitake) mushrooms (slice approximately 3 med sized mushrooms) to your desired dimensions
   h. Add ½ medium onion thinly sliced
   i. Add 1 clove of garlic finely chopped to 2 TBSP of yogurt (plain organic) and mix
   j. Add 1-2 TBSP black currants (Locate at local health food store)
   k. Add 1-2 TBSP unsweetened tart cherries (Locate at local health food store)
   l. Add 3-4 chopped dried nonsulfured Turkish apricots (Locate at local health food store)
   m. ¼ to ½ of a red bell pepper, thinly sliced
   n. Add 1 whole sliced kiwi
   o. Add 1-2 TBSP honey
   p. Sprinkle ⅛ tsp of freshly ground turmeric on salad (locate in Indian grocery)
   q. Add ½ cucumber sliced
   r. Toss this salad and eat it daily
D. Reishi and Maitake mushrooms are hard to find in a grocery store. Locate a liquid extract preparation of the Reishi mushroom (for example, Herb Pharm, Reishi 4 oz) in a health food store or online, drink a full eye drop amount 3 limes a week.

A case study was conducted on a human volunteer who presented with fungal infection, hemoptysis and HIV. After undergoing the treatment presented in the claim, the fungal infection cleared, the hemoptysis completely stopped, and the in vivo HIV 1 & 2 test was nonreactive. In just 2 months of treatment, the volunteer regained a normal life.

I claim:

1. Independent:
I claim a process for naturally eliminating HIV to be employed by type 1 and type 2 HIV suffers for 2 months which combines prayer, medical attention, reduction of energy supply to HIV by eliminating refine sugar products and exercise, abstinence to prevent re-infection, internal cleanse to detoxify body, and the combining of food antiviral, antifungal, and antibacterial properties.
A. The process that the HIV sufferer should pray to the God of Abraham or healing:
1. If the HIV sufferer hasn't participated in risky behaviors that would lead to infection, one should pray: Should I suffer as an evil doer?
2. If the HIV sufferer has participated in behavior that exposed them to the disease, one should pray: Be merciful to me, a sinner, and please heal me. Help me to go and sin no more.
B. The process that the HIV sufferer should seek physician treatment of the yeast infection with the normal fungal infection medication and, if needed, to prescribe 7-day step prednisone before embarking on the successive steps
C. The process of performing steps A, C-H daily for 2 months
D. The process of eliminating excess sugar from the diet. It is absolutely necessary to perfect this step before advancing to the remaining steps.
Eliminate the following:
1. Store bought fruit juices
2. Soda, sweetened beverages
3. Candy, cookies, brownies, donuts, pies, cakes, ice cream, flavored yogurt, bread; high glycemic foods such as potatoes, corn, etc should be kept to a minimum
E. The process of performing non-strenuous exercise
1. 20-30 minute cardio workout, aerobic breathing 5-6 days per week
2. Walk 20 30 minutes per day
3. Perform mild weight lifting 3-4 days a week, working upper and lower body on alternating days. Use exercise machines that work the upper body: chest press, triceps bar, biceps bar, and lat pull-down performing 2 sets of 10 repetitions on each machine at a light to moderate level where each set can be completed correctly without strain. On alternating days use exercise machines that work the lower body such as the seated leg press performing 3 sets of 10 and abdominal crunch machine to work the abdominals and obliques performing 2 sets of 15 on each area at a light to moderate level.
F. The process of abstaining from sexual intercourse
G. The process of performing internal cleanse to detoxify the body
1. Hydrate, drink water throughout the day (drink half your body weight in oz of water)
2. On a daily basis prior to eating, administer a water enema after eliminating waste naturally. Replace one water enema with organic coffee enema (follow steps 2.a-g except add organic coffee to the water, strain heated water through a fine mesh stainless steel strainer).
a. Heat purified water to boiling, allow to cool to approximately 98 to 105 F
b. Measure the water temperature with a candy thermometer
c. Fill an enema bag (1.6 L capacity) approximately 85% full with the heated water
d. Lubricate the probe with olive oil
e. Remove air from the tube by allowing water to flow through the tube, re-clamp
f. Lie on left side, insert probe, and modulate the fluid entry to a tolerable flow until most of the water is inside the body. Leave some water in the enema bag to prevent the introduction of air into the body
g. Allow the water to remain in the body for 20 minutes, lie on each side of the body 5 minutes—use a timer. The time introducing the water while lying on left side will usually take 2 minutes, remain on the left side for the balance of the 5 minutes, lie on your back, then your right side, and finally lie on your stomach (if too uncomfortable to lie on your stomach, bend right leg slightly and use as a prop to raise your stomach a few inches off the floor). Initially you may not be able to hold to water for the entire 20 minutes. (20 minutes is the target)
h. Eliminate the water (allow 5-15 minutes to complete the successive eliminations), and
H. The process of diet
1. Lunch and dinner should be sensible basic meals that include meat protein and low-glycemic vegetables; avoid eating from 8 p.m. to 4 a.m. unless hungry
2. Snacks (fresh fruit or raw non-starch vegetables)
3. Breakfast (it is critical that this meal is eaten daily as the first meal of the day)
a. Sprouts: Fenugreek seeds and brown lentils sprouted separately
1. Add about ⅛ cup of fenugreek seeds to a bowl and ¼ cup of brown lentils to another. Soak the seeds in water over night
2. Pour off the water and cover with a cheese cloth during the day or leave open to the air
3. Rinse the seeds every day
4. Eventually the seeds will sprout. Depending on the season this could take 3-5 days to reach the point where they are edible
5. Make new batches as needed as this is an ingredient that is needed daily. Do not over produce the sprouts because the longer the sprouted seeds grow, the more inedible they become.
b. Add a handful of fenugreek and lentil sprouts
Seeds—freshly grounded and whole
c. Freshly grounded—grind in a coffee grinder
1 TBSP of black sesame seeds and 2 TBSP of flax (unplug grinder, use a spoon to scoop out the ground seeds, add to ½ mashed avocado and mix thoroughly)
d. Whole seeds
Shelled sunflower and in-shell pumpkin seeds (approximately 1-2 TBSP each to your taste)
e. Aloe Vera Barbadensis use leaves that are whole, unbroken, no browned spots or gashes. Wash the leaf. Cut off approximately 2 inches. Cut the prickly sides off, slice it open preserving as much of the gel as possible. Cut the gel off the remaining slice, and cut it into ½ inch chunks. Add to salad.
f. Watercress leaves—add half of the bunch of pre-cleaned leaves with stems with should amount to at least a cup
g. Add fresh Shiitake (Reishi or Maitake) mushrooms (slice approximately 3 medium sized mushrooms)
h. Add ½ medium thinly sliced onion
i. Add 1 clove of garlic finely chopped to 2 TBSP of plain organic yogurt and mix
j. Add 1-2 TBSP of black currants
k. Add 1-2 TBSP unsweetened tart cherries
l. Add 3-4 chopped dried nonsulfured Turkish apricots
m. Add ¼ to ½ of thinly sliced red bell pepper
n. Add 1 whole sliced kiwi
o. Add 1-2 TBSP honey
p. Sprinkle ⅛ tsp of freshly ground turmeric on salad
q. Add ½ sliced cucumber
r. Toss this salad and eat daily
4. Drink a full eye drop of Reishi mushroom liquid extract preparation (Herb Pharm, Reishi 4 oz) 3 times a week

2. Dependent: I claim the process of eliminating the HIV virus as claimed in the independent claim 1 is sustained by the cessation of behavioral activities that lead to re-infection.
